(19) United States
(12) Patent Application Publication
(10) Pub. No.: US 2026/0155249 A1
(43) Pub. Date: Jun. 4, 2026

(54) SYSTEM AND METHOD OF TREATMENT OF HYPERTROPHIC CARDIOMYOPATHY

(71) Applicant: Prolaio, Inc., Scottsdale, AZ (US)

(72) Inventors: Jay M. Edelberg, Princeton, NJ (US); Tassos Gianakakos, Paradise Valley, AZ (US)

(21) Appl. No.: 19/122,558

(22) PCT Filed: Oct. 20, 2023
(86) PCT No.: PCT/US2023/077462

§ 371 Date: Apr. 18, 2025

Related U.S. Application Data
(60) Provisional application No. 63/380,500, filed on Oct. 21, 2022.

(51) Int. Cl. G16H 50/00

(52) U.S. Cl. CPC G16H 50/00

(57) ABSTRACT

Disclosed herein, in some aspects, are systems and methods for treating hypertrophic cardiomyopathy (HCM) in a subject. In some embodiments, the systems and methods described herein comprise monitoring a cardiac performance and optionally detecting an abnormality of said cardiac performance, in some cases after an initial HCM treatment is administered. In some cases, the initial HCM treatment includes administration of a myosin inhibitor, and/or septal myectomy. In some embodiments, said cardiac performance correlates with a right ventricular function and/or a pulmonary arterial pressure. In some embodiments, systems and methods described herein comprise identifying a therapy for a subject so as to help reduce the risk of de-performance teriorating a cardiac performance. In some cases, identifying the therapy includes predicting an effectiveness of a therapy with respect to a cardiac performance. In some embodiments, a recommendation is provided for adjusting a therapy based on an observed effect on a cardiac performance.

### CROSS-REFERENCE

This application claims the benefit of and priority to U.S. patent application Ser. No. 63/380,500, titled “System and Method of Treatment of Hypertrophic Cardiomyopathy”, and filed Oct. 21, 2022, which is incorporated herein by reference in its entirety.

### BACKGROUND

Hypertrophic cardiomyopathy (HCM) relates to a heart disease affecting the heart muscle, such as causing thickening of the heart muscle, most commonly at the septum between the right and left ventricles, which may lead to stiffness to the heart walls and changes to the mitral valve, among others, which may impede normal blood flow out of the heart. Accordingly, in some embodiments, the heart will begin to pump faster in order to provide sufficient blood to the subject, and/or in some cases, the heart will pump harder so as to overcome a pressure buildup due to the decreased blood flow. Existing HCM treatment includes providing a drug therapy so as to reduce the contractile force at which the heart pumps blood and/or reduce the speed of the heart pumping, thereby improving the overall function of the heart. Exemplary drug therapy includes administering a myosin inhibitor, one or more beta blockers, one or more calcium channel blockers, one or more heart rhythm drugs and/or one or more anticoagulants. In some cases, such as for example obstructive HCM, HCM treatment include one or more invasive measures, such as septal myectomy, septal ablation, implantable cardioverter-defibrillator, or others known in the art.

### SUMMARY

Disclosed herein in some aspects is a method for treating hypertrophic cardiomyopathy in a subject, the method comprising: detecting an abnormality with a cardiac performance in the subject, wherein the abnormality corresponds to one or more of an abnormal pulmonary arterial pressure, pulmonary hypertension, abnormal right ventricular pressure, right ventricular hypertrophy, right ventricular strain, and right ventricular size; optionally predicting an efficacy of a first therapy for reducing the abnormality; administering the first therapy or a second therapy on the subject, optionally based on the predicted efficacy of the first therapy; and monitoring the abnormality after administering the first therapy or the second therapy on the subject, and optionally detecting a negative effect.

In some embodiments, the first and/or second therapy comprises one or more types of therapy comprising myosin inhibition, a therapy for reducing pulmonary vascular resistance, one or more beta blockers, one or more calcium channel blockers, one or more heart rhythm drugs, and/or one or more blood thinners, or any combination thereof. In some embodiments, the negative effect comprises an increased abnormality in the cardiac performance from (a).

In some embodiments, predicting the efficacy of the first therapy comprises: obtaining one or more health parameters of the subject; and applying the one or more health parameters to one or more correlations relating to the first therapy. In some embodiments, the one or more correlations is based on: one or more health parameters received from a cohort of individuals; and for each individual of the cohort of individuals, an i) negative effect to a corresponding cardiac performance due to the first therapy being administered, ii) a positive result to the corresponding cardiac performance due to the first therapy being administered, or iii) no effect to the corresponding cardiac performance due to the first therapy being administered, such that at least one of the one or more health parameters (from a corresponding individual of the cohort of individuals) is correlated with an effect on a cardiac performance when combined with administration of the first therapy. In some embodiments, applying the one or more correlations of data comprises using a machine learning algorithm.

In some embodiments, detecting the abnormality and/or monitoring the abnormality comprises using an implanted pulmonary arterial monitor, obtaining an echocardiogram, obtaining an electrocardiogram (ECG), obtaining one or more biomarkers, or a combination thereof. In some embodiments, detecting the abnormality and/or monitoring the abnormality is conducted in a medical setting, in an ambulatory setting, or both. In some embodiments, detecting the abnormality and/or monitoring the abnormality comprises obtaining an ECG, wherein the ECG comprises a single lead ECG, a 2 lead ECG, a 6 lead ECG, or a 12 lead ECG.

In some embodiments, the method further comprising adjusting the first or second therapy based on detecting a corresponding negative effect or corresponding no effect on the abnormality in the cardiac performance. In some embodiments, adjusting the first or second therapy comprises administering a third therapy, and/or reducing an amount of the first or second therapy administered. In some embodiments, reducing the amount of the first or second therapy administered comprises reducing a frequency of the first or second therapy is administered, and/or reducing a dosage of the first or second therapy administered. In some embodiments, prior to detecting the cardiac performance abnormality, further comprising administering an initial HCM treatment on the subject. In some embodiments, the initial HCM treatment comprises administering a myosin inhibitor to the subject.

In some embodiments, the hypertrophic cardiomyopathy comprises an obstructive hypertrophic cardiomyopathy (oHCM). In some embodiments, prior to detecting the cardiac performance abnormality, further comprising at least partially removing an obstruction relating to the oHCM. In some embodiments, the at least partially removing the obstruction comprises performing a septal myectomy.

Disclosed herein, in some aspects, is a non-transitory computer readable medium for treating hypertrophic cardiomyopathy in a subject, the non-transitory computer readable medium comprising instructions that, when executed by a processor, cause the processor to perform operations including: detecting an abnormality with a cardiac performance in the subject, wherein the abnormality corresponds to one or more of an abnormal pulmonary arterial pressure, pulmonary hypertension, abnormal right ventricular pressure, right ventricular hypertrophy, right ventricular strain, and right ventricular size; optionally predicting an efficacy of a first therapy for reducing the abnormality; determining a treatment comprising an administration of the first therapy or a second therapy on the subject, optionally based on the predicted efficacy of the first therapy; and monitoring the abnormality after administering the first therapy or the second therapy on the subject, and optionally detecting a negative effect.

In some embodiments, the first and/or second therapy comprises one or more types of therapy comprising myosin inhibition, a therapy for reducing pulmonary vascular resistance, one or more beta blockers, one or more calcium channel blockers, one or more heart rhythm drugs, and/or one or more blood thinners, or any combination thereof. In some embodiments, the negative effect comprises an increased abnormality in the cardiac performance from (a).

In some embodiments, predicting the efficacy of the first therapy comprises: obtaining one or more health parameters of the subject; and applying the one or more health parameters to one or more correlations relating to the first therapy. In some embodiments, the one or more correlations is based on: one or more health parameters received from a cohort of individuals; and for each individual of the cohort of individuals, an i) negative effect to a corresponding cardiac performance due to the first therapy being administered, ii) a positive result to the corresponding cardiac performance due to the first therapy being administered, or iii) no effect to the corresponding cardiac performance due to the first therapy being administered, such that at least one of the one or more health parameters (from a corresponding individual of the cohort of individuals) is correlated with an effect on a cardiac performance when combined with administration of the first therapy. In some embodiments, applying the one or more correlations of data comprises using a machine learning algorithm.

In some embodiments, detecting the abnormality and/or monitoring the abnormality comprises obtaining one or more cardiac parameters of the subject using an implanted pulmonary arterial monitor, obtaining an echocardiogram, obtaining an electrocardiogram (ECG), obtaining one or more biomarkers, or a combination thereof. In some embodiments, detecting the abnormality and/or monitoring the abnormality is conducted in a medical setting, in an ambulatory setting, or both. In some embodiments, detecting the abnormality and/or monitoring the abnormality comprises obtaining an ECG, wherein the ECG comprises a single lead ECG, a 2 lead ECG, a 6 lead ECG, or a 12 lead ECG.

In some embodiments, the method further comprising determining an adjustment of the first or second therapy based on detecting a corresponding negative effect or corresponding no effect on the abnormality in the cardiac performance. In some embodiments, the adjustment of the first or second therapy comprises determining an administration of a third therapy, and/or reducing an amount of the first or second therapy administered. In some embodiments, reducing the amount of the first or second therapy administered comprises reducing a frequency of the first or second therapy is administered, and/or reducing a dosage of the first or second therapy administered.

In some embodiments, prior to detecting the cardiac performance abnormality, an initial HCM treatment is administered on the subject. In some embodiments, the initial HCM treatment comprises administering a myosin inhibitor to the subject. In some embodiments, the hypertrophic cardiomyopathy comprises an obstructive hypertrophic cardiomyopathy (oHCM). In some embodiments, prior to detecting the cardiac performance abnormality, an obstruction relating to the oHCM is at least partially removed. In some embodiments, the obstruction is at least partially removed via a septal myectomy.

Disclosed in some aspects, is a system for treating hypertrophic cardiomyopathy in a subject, the subject comprising: one or more processors; and one or more memories storing instructions that, when executed by the one or more processors, cause the system to perform operations including: detecting an abnormality with a cardiac performance in the subject, wherein the abnormality corresponds to one or more of an abnormal pulmonary arterial pressure, pulmonary hypertension, abnormal right ventricular pressure, right ventricular hypertrophy, right ventricular strain, and right ventricular size; optionally predicting an efficacy of a first therapy for reducing the abnormality; determining a treatment comprising an administration of the first therapy or a second therapy on the subject, optionally based on the predicted efficacy of the first therapy; and monitoring the abnormality after administering the first therapy or the second therapy on the subject, and optionally detecting a negative effect.

In some embodiments, the first and/or second therapy comprises one or more types of therapy comprising myosin inhibition, a therapy for reducing pulmonary vascular resistance, one or more beta blockers, one or more calcium channel blockers, one or more heart rhythm drugs, and/or one or more blood thinners, or any combination thereof.

In some embodiments, the negative effect comprises an increased abnormality in the cardiac performance from (a). In some embodiments, predicting the efficacy of the first therapy comprises: obtaining one or more health parameters of the subject; and applying the one or more health parameters to one or more correlations relating to the first therapy. In some embodiments, the one or more correlations is based on: one or more health parameters received from a cohort of individuals; and for each individual of the cohort of individuals, an i) negative effect to a corresponding cardiac performance due to the first therapy being administered, ii) a positive result to the corresponding cardiac performance due to the first therapy being administered, or iii) no effect to the corresponding cardiac performance due to the first therapy being administered, such that at least one of the one or more health parameters (from a corresponding individual of the cohort of individuals) is correlated with an effect on a cardiac performance when combined with administration of the first therapy. In some embodiments, applying the one or more correlations of data comprises using a machine learning algorithm.

In some embodiments, detecting the abnormality and/or monitoring the abnormality comprises obtaining one or more cardiac parameters of the subject using an implanted pulmonary arterial monitor, obtaining an echocardiogram, obtaining an electrocardiogram (ECG), obtaining one or more biomarkers, or a combination thereof. In some embodiments, detecting the abnormality and/or monitoring the abnormality is conducted in a medical setting, in an ambulatory setting, or both. In some embodiments, detecting the abnormality and/or monitoring the abnormality comprises obtaining an ECG, wherein the ECG comprises a single lead ECG, a 2 lead ECG, a 6 lead ECG, or a 12 lead ECG.

In some embodiments, the method further comprising determining an adjustment of the first or second therapy based on detecting a corresponding negative effect or corresponding no effect on the abnormality in the cardiac performance. In some embodiments, the adjustment of the first or second therapy comprises determining an administration of a third therapy, and/or reducing an amount of the first or second therapy administered. In some embodiments, reducing the amount of the first or second therapy administered comprises reducing a frequency of the first or second therapy is administered, and/or reducing a dosage of the first or second therapy administered.

In some embodiments, prior to detecting the cardiac performance abnormality, an initial HCM treatment is administered on the subject. In some embodiments, the initial HCM treatment comprises administering a myosin inhibitor to the subject.

In some embodiments, the hypertrophic cardiomyopathy comprises an obstructive hypertrophic cardiomyopathy (oHCM). In some embodiments, prior to detecting the cardiac performance abnormality, an obstruction relating to the oHCM is at least partially removed. In some embodiments, the obstruction is at least partially removed via a septal myectomy.

### BRIEF DESCRIPTION OF THE DRAWINGS

These and other features, aspects, and advantages of some embodiments will become better understood with regard to the following description and accompanying drawings.

FIG. 1 depicts an exemplary flow chart of a method for a treatment for HCM, according to an embodiment described herein.

FIG. 2 depicts an exemplary system flow chart for a treatment for HCM, according to an embodiment described herein.

FIG. 3A depicts a block diagram of the cardiac performance tool, in accordance with an embodiment.

FIG. 3B depicts a block diagram of exemplary clinical data parameters, in accordance with an embodiment.

FIG. 4 depicts an exemplary computer system, in accordance with an embodiment.

FIG. 5 depicts exemplary data correlating elevated pulmonary arterial pressure with HCM.

FIG. 6 depicts exemplary data showing that elevated pulmonary arterial pressure does not necessarily depend in variations with the LVOT gradient pressure.

FIG. 7 depicts exemplary data depicting pulmonary arterial pressure before, shortly after, and much longer after a septal reduction therapy.

FIG. 8 depicts an exemplary flow chart depicting the cause and effect due to changes in certain cardiac conditions.

### DETAILED DESCRIPTION

### I. Definitions

Terms used in the claims and specification are defined as set forth below unless otherwise specified.

The terms “subject” or “patient” are used interchangeably and encompass a cell, tissue, or organism, human or non-human, whether in vivo, ex vivo, or in vitro, male or female.

The terms “treating,” “treatment,” or “therapy” may be used interchangeably.

It must be noted that, as used in the specification, the singular forms “a,” “an” and “the” include plural referents unless the context clearly dictates otherwise.

The terms “ambulatory”, “ambulatory measurement”, “ambulatory monitoring”, “ambulatory monitoring parameters,” etc. refer to obtaining health data (e.g., subject health parameters, as described herein) outside a hospital or other medical location (e.g., outside a medical clinic). For example, ambulatory monitoring may refer to monitoring health data (e.g., electrocardiogram, blood pressure, weight, etc.) at home.

The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e., “one or the other but not both”) when preceded by terms of exclusivity, such as “either,” “one of,” “only one of,” or “exactly one of.”

### II. HCM Treatment Overview

Described herein, in some embodiments, are systems and methods for treating hypertrophic cardiomyopathy (HCM) in a subject. In some embodiments, the systems and methods described herein are provided in conjunction with an initial HCM treatment administered. In some embodiments, the systems and methods described herein comprise monitoring a cardiac performance and optionally detecting an abnormality of said cardiac performance. In some embodiments, said cardiac performance correlates with a right ventricular function and/or a pulmonary arterial pressure (PAP), which relates to the delivery of deoxygenated blood through the lungs where it is oxygenated and then flows to the left atrium/ventricle for delivery of oxygenated blood to the body. In some embodiments, systems and methods described herein comprise identifying a therapy for a subject so as to help reduce the risk of deteriorating a cardiac performance, wherein a deteriorated cardiac performance may result in a reduced cardiac output (by the heart) for delivering sufficient blood to the subject's body, and/or may result in the development of systolic dysfunction and related heart failure. In some embodiments, such identification of a therapy comprises predicting an effectiveness of a therapy with respect to a cardiac performance. In some embodiments, systems and methods described herein comprise providing a recommendation for adjusting a therapy based on an observed effect on a cardiac performance (e.g., effect on right ventricular function, PAP, etc.).

As described herein, in some embodiments, systems and methods described herein for treating HCM are based on monitoring a cardiac performance and detecting any abnormalities therewith. As used herein, an abnormality in the cardiac performance refers to subpar parameters relating to the heart operation. For example, the right ventricular function (RVF) drives deoxygenated blood through the lungs for oxygenation and to the left atrium/left ventricle for delivery throughout the body. Accordingly, a build-up of pressure resistance caused by HCM, and/or a reduction in blood flow due to HCM, may result in the right ventricle to increase its contractile force, and/or pump with more force so as to overcome the pressure resistance build-up and increase the blood flow supplied to the body respectively. In some embodiments, such increase in contractile force and pumping force by the right ventricle correlates with a reduction in the right ventricle function (RVF). Accordingly, such reduction in right ventricle function correlates in a reduced cardiac performance, or an abnormality with the cardiac performance. In this example, an increase in right ventricular contractile force may result in right ventricle hypertrophy, a known heart disorder, and which may result in cardiac output and/or systolic dysfunction.

In some embodiments, the cardiac performance (as described herein) is determined using one or more cardiac parameters measured from the subject. For example, in some embodiments, the cardiac performance corresponds to one or more of pulmonary arterial pressure (PAP) (including the presence of pulmonary hypertension), pulmonary vascular resistance, right ventricular pressure (systolic and/or diastolic), right ventricular hypertrophy (e.g., enlargement of right ventricle, or increase in muscle mass), right ventricular strain, and right ventricular size. In some embodiments, an abnormal reading in any one or any combination of such cardiac parameters correlates an abnormality in the cardiac performance, and therefore a reduction in the cardiac performance. In some embodiments, such abnormal reading for said cardiac parameters includes a higher measured value compared to a standard range. For example, in some cases, a normal PAP for a subject ranges from about 8 to 20 mmHg at rest, such that a higher PAP from this range may correlate to an abnormality in the cardiac performance and may be an indication of pulmonary hypertension. In some cases, a normal pulmonary vascular resistance is less than about 2 Wood units (WU), such that an increased resistance above 3 WU may correlate to an abnormality in the cardiac performance. In some embodiments, the right ventricular pressure includes one or both of the right ventricular systolic pressure and the right ventricular diastolic pressure. In some cases, a normal right ventricular systolic pressure (relating to contractile force) ranges from about 15 to 30 mmHg, and a normal right ventricular diastolic pressure (relating to filling of the right ventricle) ranges from about 1 to 7 mmHg, such that an increased pressure from one or both of these ranges may correlate to an abnormality in the cardiac performance. In some embodiments, a normal right ventricle size may range from about 49 to 101 ml/m2 , compared to the left ventricle which may range from about 44 to 80 ml/m2 , such that an increased size from the normal right ventricular size range and/or an in proportional to the left ventricle may correlate to an abnormality in the cardiac performance.

In some embodiments, the further a measured cardiac parameter reading is from a respective normal range corresponds to a more abnormal or more poor cardiac performance. In some cases, the more abnormal or more poor a cardiac performance exists, the higher the risk for the heart to fall below a sufficient cardiac output and/or the higher the risk of the heart developing systolic dysfunction.

In some embodiments, the cardiac performance is based on a combination of one or more cardiac parameters. For example, in some cases, an increased PAP requires an increased right ventricular force in order for the heart to maintain a certain cardiac output. Accordingly, although in some cases an increased right ventricular force correlates with a cardiac performance abnormality, an increased PAP with a lower right ventricular force may be a more severe abnormality than an increased PAP with a higher right ventricular force, since a lower right ventricular force may result in a reduction in cardiac output.

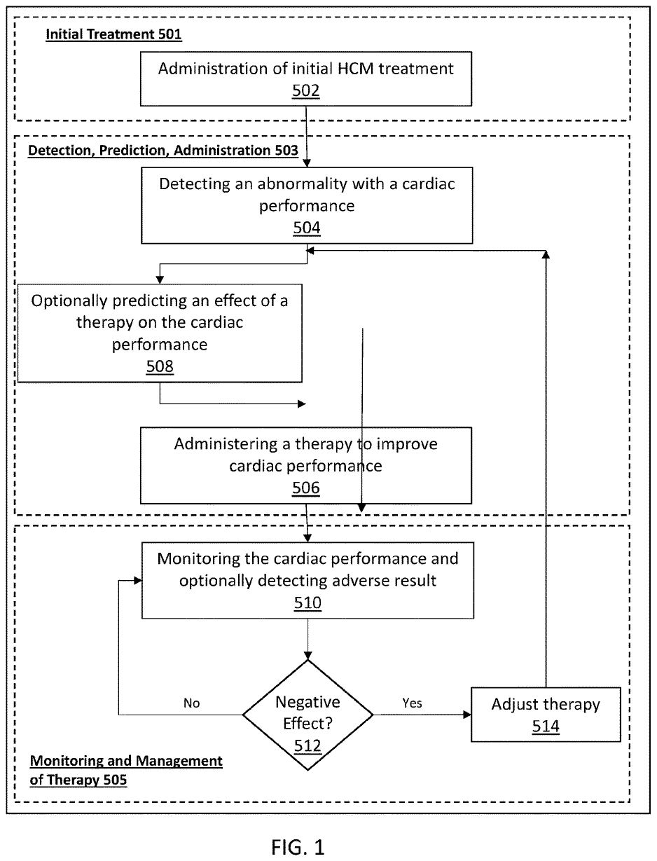

FIG. 1 (FIG. 1) depicts an exemplary flowchart for a method for treating HCM in a subject, as described herein. In some embodiments, the treatment comprises one or more stages, any one of which can be administered singularly or in combination with another stage to provide said treatment for HCM.

In some embodiments, a first stage for treatment 501 comprises administrating an initial treatment for HCM 502, as described herein. In some embodiments, the initial HCM treatment administered on the subject comprises a treatment as currently known in the art.

For example, in some embodiments, an initial treatment for HCM includes performing septal myectomy (for obstructive HCM) on the subject, or performing septal ablation. Other forms of initial HCM treatment that may or may not be administered in combination with septal myectomy or septal ablation include providing a drug therapy, such as administering a myosin inhibitor, a beta blocker, a calcium channel blocker, a heart rhythm drug and/or one or more anticoagulants.

In some embodiments, a second stage of a treatment 503 described herein comprises monitoring the cardiac performance of the subject and identifying the subject as having a risk for cardiac output reduction and/or systolic dysfunction, among other heart failures, based on detection 504 of a cardiac performance abnormality in the subject. In some embodiments, detecting said abnormality with the cardiac performance is based on measuring one or more cardiac parameters, as described herein. In some embodiments, measuring said one or more cardiac parameters is via an implanted pulmonary arterial monitor, an echocardiogram, an electrocardiogram (ECG), one or more biomarkers indicative of a cardiac performance state, or a combination thereof. As used herein, the cardiac performance state refers to a status of the cardiac performance as measured via one or more cardiac parameters, and includes an abnormality with the cardiac performance, a proper or healthy cardiac performance. In some embodiments, said one or more cardiac parameters are measured in a clinical setting, such as at a medical office, hospital, lab, etc. In some embodiments, said one or more cardiac parameters are obtained in an ambulatory setting, for example via an ECG device from a subject's household or other non-clinical environment. As described herein under the System Overview section, in some embodiments, such ambulatory monitoring may be in communication with a system configured to monitor the cardiac performance, provide communication to a healthcare provider (e.g., physician, other medical professional), and/or present one or more recommendations to the subject based on the cardiac performance reading, wherein such recommendations may be automatically generated by the system and/or provided via a healthcare provider.

Accordingly, in some embodiments, the second stage treatment 503 comprises correlating a cardiac performance with an initial HCM treatment (e.g., from step 502). For example, in some embodiments, after an initial HCM treatment (e.g., administering a myosin inhibitor) is provided to the subject, the cardiac performance of the subject is then monitored to determine whether there exists an abnormality (e.g., a continued abnormality), and whether such abnormality is increasing (e.g., whether the cardiac performance is worsening or becoming more abnormal).

For example, in some embodiments, a septal myectomy is performed (e.g., as an initial HCM treatment) to remove an obstruction (i.e., obstructive HCM), thereby helping at least reduce a left ventricle outflow tract (LVOT) gradient pressure. In some cases, however, a subject having a septal myectomy may continue to experience high pulmonary arterial pressures (PAP) for a time period after the procedure, such as for example one year.

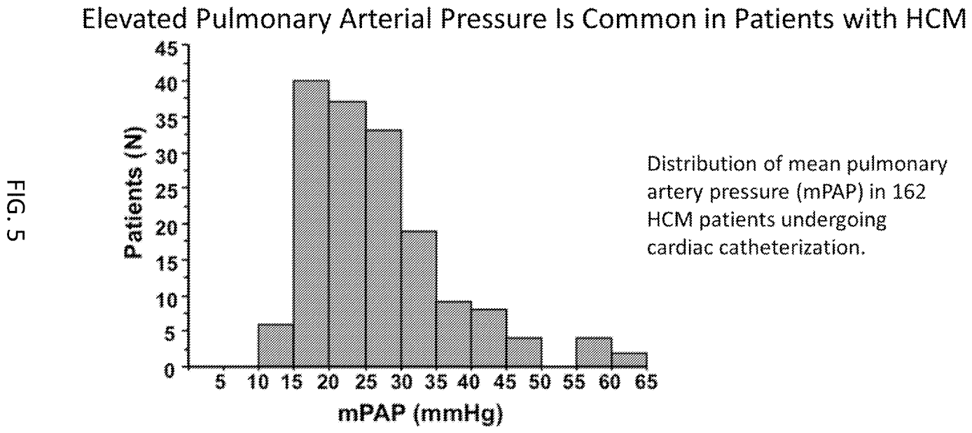

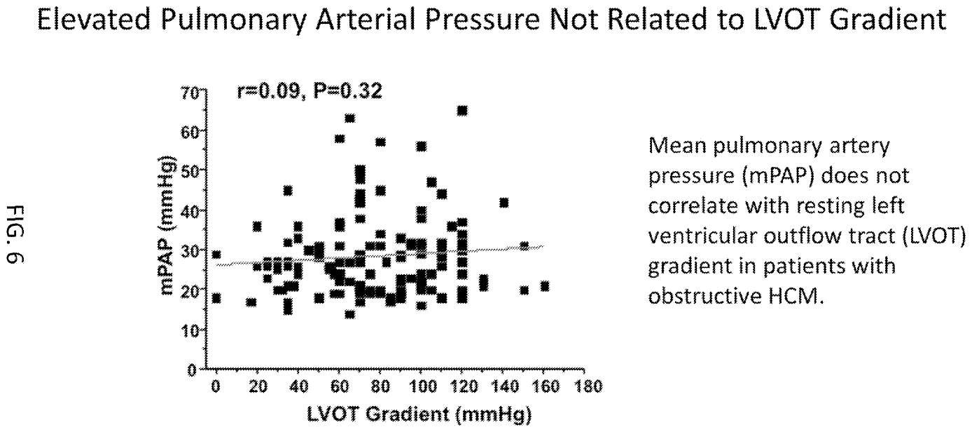

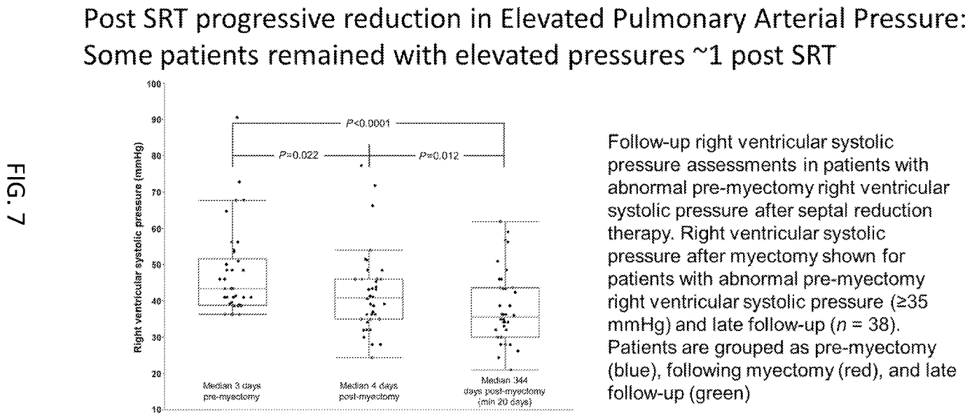

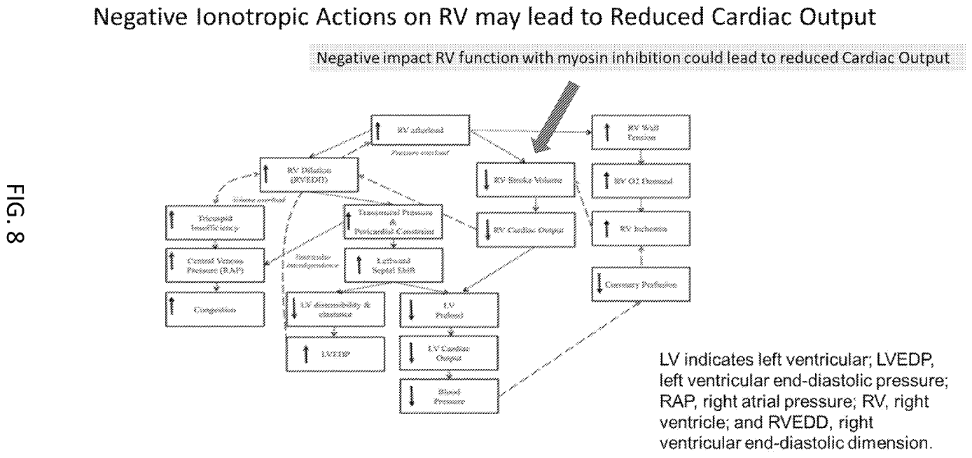

Accordingly, in such cases, an increased right ventricular contractile force is needed so as to maintain sufficient cardiac output (sufficient blood flow for the body) and/or help prevent or reduce the risk of systolic dysfunction from occurring. See for example FIGS. 5-7 wherein FIG. 5 depicts exemplary data that shows elevated PAP is common in patients with HCM (reference: Circ Heart Fail. 2017 Apr; 10(4): e003689; doi: 10.1161/CIRCHEARTFAILURE.116.003689), FIG. 6 depicts that elevated PAP does not necessarily correlate with LVOT gradient pressure (i.e., reduction or increase in LVOT gradient pressure may not impact PAP) (reference: Circ Heart Fail. 2017 Apr; 10(4): e003689; doi: 10.1161/CIRCHEARTFAILURE.116.003689), and FIG. 7 depicts exemplary data that shows some cases where elevated PAP remained after a septal reduction therapy (reference: Eur Heart J, Volume 35, Issue 30, 7 Aug. 2014, Pages 2032-2039, https: //doi.org/10.1093/eurheartj/eht537).
In another exemplary embodiment, a drug therapy is administered as part of the initial HCM treatment, such as a myosin inhibitor, so as to help reduce the LVOT gradient pressure. Therefore, similar to as described with septal myectomy, the PAP may remain elevated for a duration period of time. In some cases however, the myosin inhibitor helps reduce the contractile force of the heart, including RV contractile force. Accordingly, in some cases, though the administration of a myosin inhibitor helps reduce the LVOT gradient pressure (that resulted due to the HCM), the reduction in RV contractile force may impact the ability in the RV to overcome the increased PAP that may remain, thereby potentially impacting the ability of the heart to maintain a cardiac output (e.g., blood flow to the body), and/or may result in systolic dysfunction. For example, see FIG. 8 depicting an exemplary cause and effect flow chart relating to the interrelation between various cardiac components (reference: Marvin A. Konstam. Circulation. Evaluation and Management of Right-Sided Heart Failure: A Scientific Statement From the American Heart Association, Volume: 137, Issue: 20, Pages: e578-e622, DOI: (10.1161/CIR.0000000000000560). As depicted in FIG. 8, administration of myosin inhibitor may lead to decreased right ventricle stroke volume, thereby lowering the right ventricle cardiac output.

In some embodiments, such monitoring of the cardiac performance helps alert the subject or other personnel (e.g., a healthcare provider) of an unintended deterioration in cardiac performance after an initial HCM treatment. In some embodiments, a therapy is administered to improve cardiac performance. In some embodiments, such therapy comprises providing a drug therapy to the subject, and/or a surgical procedure. For example, in some embodiments, one or more of a myosin inhibitor, a beta blocker, a calcium channel blocker, a heart rhythm drug, and a blood thinner is provided so as to help reduce the cardiac performance abnormality. For example, in some cases, wherein a myosin inhibitor is provided as an initial HCM treatment, and wherein a cardiac performance abnormality persists or worsens (e.g., due to the reduced contractile force of the right ventricle), one or more therapies to reduce pulmonary vascular resistance (e.g., a PDE5 inhibitor) may be provided so as reduce the pressure needed to deliver the blood from the right ventricle.

Accordingly, this will help offset the reduction in contractile force by the right ventricle via the myosin inhibitor.

In some embodiments, an effect of a therapy administered to a subject is first predicted 508, to help reduce the risk of the subject experiencing a negative effect (e.g., a negative effect on the cardiac performance) due to the therapy. In some embodiments, one or more subject health parameters, which may include the one or more cardiac parameters described herein, are used to help predict said effect of a therapy (as described under the System Overview section). For example, in cases where PAP is detected to be high in an individual, it may be predicted that administration of a myosin inhibitor may result in an increased risk for reduced cardiac output and/or systolic dysfunction.

In some embodiments, as described herein, said effect of a therapy is based on one or more subject health parameters (which may include the one or more cardiac parameters described herein), as described herein under the system overview section, wherein exemplary subject health parameters are listed. In some embodiments, said predicted effect of a therapy is determined based on one or more correlations relating the one or subject health parameters with an effect of a given therapy (as described herein under the system overview). In some embodiments, such correlations are based on data received from a plurality of individuals (e.g., a cohort of individuals), where such data includes, from each individual, one or more subject health parameters, a therapy administered, and the resulting effect on a cardiac performance as described herein. For example, in some embodiments, the resulting effect corresponds to a reduced cardiac performance (e.g., a negative effect), an improved cardiac performance (e.g., a positive effect), or no change to the cardiac performance (e.g., no effect). In some embodiments, said correlations are developed using a machine learning algorithm (as described herein). Accordingly, in some embodiments, a system described herein is used to predict an effect of a therapy on the cardiac performance of a subject, which may include the use of a machine learning algorithm.

With reference to step 508 of FIG. 1, in some embodiments, if a negative effect is identified with a therapy, another therapy is then evaluated. In some embodiments, using a system as described herein, one or more therapies are suggested by a healthcare professional (e.g., a physician, other medical professional) for the system to evaluate and predict the effect on the cardiac performance. In some embodiments, as described herein, the system automatically determines one or more therapies to evaluate based on the determined cardiac performance and subject health parameters. In some embodiments, the therapy administered at step 506 is based on the therapy identified as predicting to have a positive effect, or at least no effect.

In other embodiments, as described herein, an initial HCM treatment is not administered on the subject, and thus the step 504 is initially performed on a subject to identify a cardiac performance abnormality, after which a therapy is optionally identified 508, and administered 506. In some embodiments, such therapy includes any therapy that was identified with an initial HCM treatment herein (e.g., septal myectomy, myosin inhibitor, etc.). In some embodiments, a therapy comprises one or more of various therapies, as described herein, such a myosin inhibitor and a PDE5 inhibitor or a beta blocker, etc.

In some embodiments, the third stage 505 of a HCM treatment described herein comprises monitoring 510 the cardiac performance after administering the therapy from the second stage 503 (e.g., step 506). In some embodiments, monitoring the cardiac performance comprises using any means as described herein, such as at step 504, which includes measuring one or more cardiac parameters, as described herein. In some embodiments, measuring said one or more cardiac parameters is via an implanted pulmonary arterial monitor, an echocardiogram, an electrocardiogram (ECG), one or more biomarkers indicative of a cardiac performance state, or a combination thereof. In some embodiments, said one or more cardiac parameters are measured in a clinical setting, such as at a medical office, hospital, lab, etc. In some embodiments, said one or more cardiac parameters are obtained in an ambulatory setting, for example via an ECG device from a subject's household or other non-clinical environment.

In some embodiments, using a system described herein, ambulatory monitoring allows for periodic measurements of one or more cardiac parameters, so as to continually monitor the subject for a period of time without the need to visit a medical office or clinic. In some embodiments, using a system as described herein, such monitoring includes communicating to the subject and/or a healthcare provider the cardiac performance state, wherein progression or regression of an abnormality can be monitored.

In some embodiments, such monitoring 510 identifies 512 a negative effect on the cardiac performance based on a rate of increase in abnormality (e.g., based on an increase in deviation of one or more cardiac parameters, singularly or in combination, from a normal range, as described herein). In some embodiments, after detecting a negative effect on a cardiac performance, a recommendation is provided, for example, by the system and/or a personnel (e.g., subject, healthcare professional), to adjust the therapy 514 administered. In some embodiments, adjusting the therapy comprises maintaining the same therapy, but changing one or more of the therapy dosage and the frequency. For example, where a myosin inhibitor is provided, in some embodiments, adjusting the therapy comprises providing a reduced amount of the myosin inhibitor, and/or reducing the frequency of when the myosin inhibitor is provided.

In some embodiments, adjusting the therapy comprises changing one or more of the therapy dosage and the frequency, and/or providing one or more additional therapies. For example, where a myosin inhibitor is provided and found to reduce the contractile force of the right ventricle while the PAP remains elevated, a PDE5 inhibitor or other therapy to decrease the pulmonary vascular resistance is recommended to be administered to the subject.

In some embodiments, adjusting the therapy comprises to stop providing the existing therapy, and recommend one or more additional therapies to be provided, or alternatively, not provide any therapy (either for a prescribed duration, or stop completely).

In some embodiments, providing one or more additional therapies, either with or without the existing therapy (from step 506), includes first predicting the effect of the therapy prior to either recommending or administering to the subject (via step 508). In some embodiments, where a negative effect to the cardiac performance is detected, or in some cases, a positive effect is not identified, the method re-starts after step 504.

In some embodiments, wherein a negative effect is not identified, or in some cases, if a positive effect is identified 512, the cardiac performance of the subject is continued to be monitored 510.

### III. System Overview

In some embodiments, one or more steps of a method described herein are performed using a system described herein. For example, in some embodiments, stages 2 and 3 of the HCM treatment method described in FIG. 1 is at least partially performed using a system described herein.

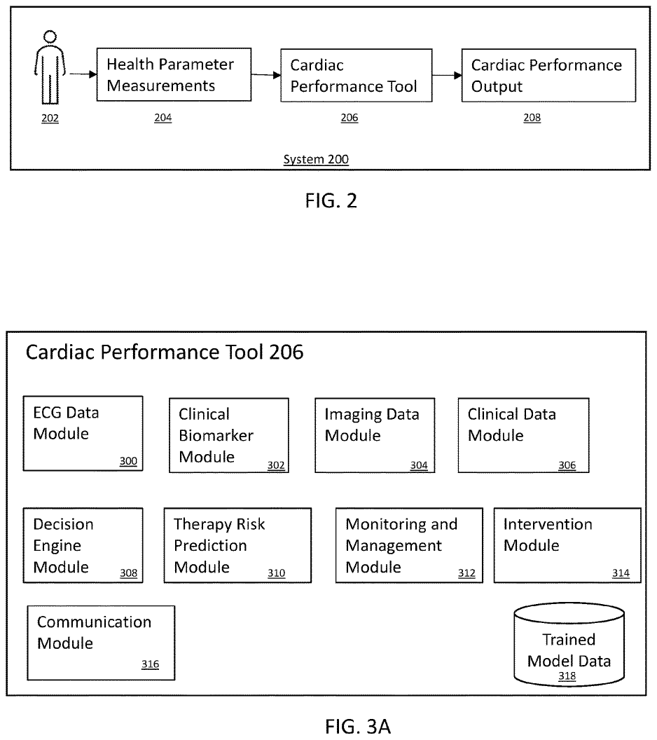

FIG. 2 depicts an overview of an exemplary system 200 for detecting, monitoring, and managing a cardiac performance abnormality for a subject 202. In some embodiments, the system 200 receives subject health parameter measurements 204 from one or more devices that are then used by a cardiac performance tool (CPT) 206 to produce a cardiac performance output, which may include a cardiac performance state 208, a prediction of an effect of a therapy on a cardiac performance state, and a recommended therapy for reducing a cardiac abnormality.

In some embodiments, the system 200 provides an integrated management tool for detecting, monitoring, and managing a cardiac performance abnormality for the subject, and for communicating to the subject and/or a healthcare provider (e.g., physician, nurse, or any other medical professional) the cardiac performance monitoring, alerts, and/or recommendations.

With reference to FIG. 2, as described herein, the subject health parameter measurements 204 for a subject 202 (e.g., ECG data from an ECG device) are received by a cardiac performance tool 206, which then generates an output relating to a cardiac performance 208 for the subject. In some embodiments, the cardiac performance output is communicated onto a display interface (e.g., a monitor, screen, smart device screen, etc.). As described herein, in some embodiments, the subject health parameter measurements 204 are obtained via a device or inputted (into a computing device, which may include the system 200) by an individual (e.g., subject, healthcare provider, subject's family/friend, other individual). In some embodiments, wherein the subject health parameter measurements are obtained via a device, said device and the cardiac performance tool 206 are used by different parties. For example, a first party (for example, the subject 202, a medical professional, or any other person) operates an ECG device to obtain the ECG data from the subject 202 (e.g., health parameter measurement 204), wherein the ECG data is then provided to a second party (for example, the subject 202, a medical professional, or any other person different from the individual operating the ECG device) which implements the cardiac performance tool 206 to determine a cardiac performance output 208. In some embodiments, the device to obtain the health parameter measurement 204 and the cardiac performance tool 206 are used by the same party. Similarly, in some embodiments, wherein the health parameter measurements are obtained via being inputted into a computing device, said inputting and operating the cardiac performance tool 206 are performed by the same party or by different parties.

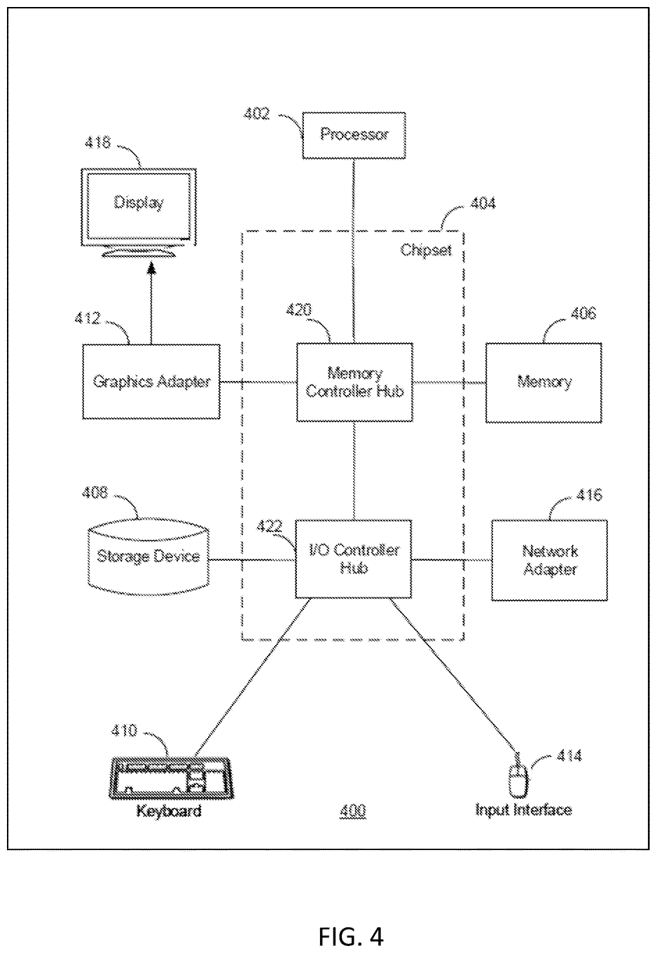

In some embodiments, the cardiac performance tool is provided by one or more computing devices, wherein the cardiac performance tool can be embodied as a computer system (e.g., see FIG. 4, reference character 400). Accordingly, in some embodiments, methods and steps described in reference to the cardiac performance tool 206 are performed in silico. For example, in some embodiments, the cardiac performance tool is configured to apply one or more health parameter measurements to one or more decision engines (e.g., trained models, decision trees, analytical expressions, etc.) so as to predict an efficacy of a therapy for reducing a cardiac performance abnormality in a subject. In some embodiments, the one or more decision engines each apply an algorithm, such as a machine learning algorithm (as described herein), to the one or more health parameter measurements.

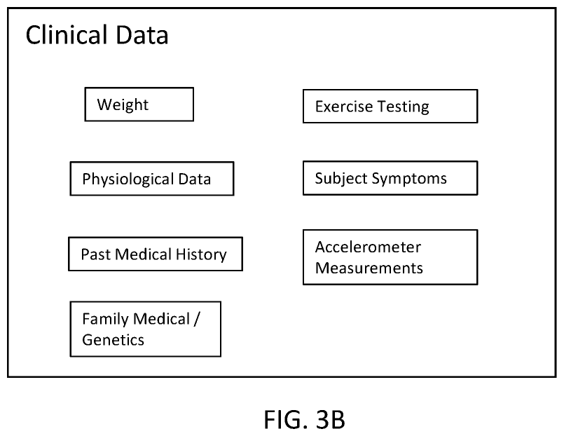

With reference to FIG. 3A, a block diagram is depicted illustrating exemplary computer logic components of the cardiac performance tool 206, in accordance with an embodiment. Here, the cardiac performance tool 206 includes an ECG data module 300, a clinical biomarker module 302, an imaging data module 304, a clinical data module 306, a decision engine module 308, a therapy risk prediction module 310, a monitoring and management module 312, an intervention module 314, a communication module 316, and a decision engine data storage 318. In some embodiments, the cardiac performance tool 206 can be configured differently with additional or fewer modules. For example, a cardiac performance tool 206 need not include the imaging data module 304. In some embodiments, the decision engine module 308 and/or the decision engine data storage 318 are located on a different tool and/or computing device.

As described herein, in some embodiments, the cardiac performance tool 206 is configured to determine a cardiac performance output 208 for a subject 202, which may include determining a cardiac performance state and/or to predict an efficacy of a therapy for reducing a cardiac performance abnormality. In some embodiments, the cardiac performance tool 206 applies subject health parameters obtained for the subject to one or more decision engines to determine the cardiac performance state, and/or to determine the efficacy of a treatment for reducing a cardiac performance abnormality. In some embodiments, the subject's health parameters are obtained via the ECG data module 300, the clinical biomarker module 302, the imaging data module 304, and/or the clinical data module 306.

### Subject Health Parameters

In some embodiments, the subject health parameters, as described herein, comprise one or more cardiac parameters, one or more ECG parameters, one or more clinical biomarkers, one or more imaging data (e.g., echocardiogram, MRI), and/or one or more clinical data.

In some embodiments, the ECG data module 300 is configured to be in operative communication with an ECG device for obtaining ECG data from the subject. Accordingly, in some embodiments, the ECG data module is configured to receive ECG data from the ECG device, and optionally extract one or more parameters of the ECG data. The ECG device can be any device known in the art used for obtaining ECG data. For example, in some embodiments, the ECG device comprises a 12-lead ECG device, a 6-lead ECG device, a single lead ECG device, or a double lead ECG device. In some embodiments, the 12-lead ECG device is found at a health location, such as a healthcare provider office or clinic, including a hospital, physician's office, medical clinic, or any other location staffing medical and/or health professionals (e.g., physicians, emergency medical technicians, nurses, first responders, psychologists, phlebotomist, medical physics personnel, nurse practitioners, surgeons, dentists, and any other obvious medical professional as would be known to one skilled in the art). In some embodiments, the ECG device is configured to be used outside a healthcare provider's office or clinic (as described herein). For example, the ECG device can be used by the subject at home (e.g., ambulatory monitoring). In some embodiments, the ECG device, for example a single or double lead ECG device, is incorporated in a wearable device (for example a watch, smartwatch) or other type of mobile device, and thus configured to obtain ECG data while the subject is at rest and/or moving. As used herein, the term “ambulatory monitoring” or “ambulatory measurements” refer to monitoring that occurs and/or measurements that are obtained outside a medical location, such as a hospital or other type of medical location (e.g., a clinic).

In some embodiments, the ECG device (for example a wearable device such as a smartwatch) incorporates its own software (such as a software application, or an “app”) to store the raw ECG data obtained by the ECG device, which may include, without limitation, ECG waveforms. In some embodiments, the ECG device software is in operative communication with the ECG data module 300. In some embodiments, the ECG data module 300 includes the software application receiving the raw ECG data.

In some embodiments, the ECG data module is configured to extract specific parameters from the ECG data for determining a cardiac performance state and/or predicting an efficacy of a therapy. In some embodiments, the one or more specific ECG parameters that can be extracted from the ECG data include P wave parameters, PR interval parameters, QRS complex, J-point, ST segment, T wave, corrected QT interval, U wave, or any combination thereof. In some embodiments, the ECG data module is configured to correlate one or more ECG parameters with one or more cardiac parameters for detecting a cardiac performance state.

In some embodiments, the ECG data module 300 provides one or more of the ECG parameters to the therapy risk prediction module 310 and/or the monitoring and management module 312. In some embodiments, the therapy risk prediction module 310 and/or the monitoring and management module 312 are configured to extract the one or more ECG parameters from the ECG data received by the ECG data module 300. In some embodiments, the therapy risk prediction module 310 corelates one or more ECG parameters with one or more subject health parameters to determine an efficacy of a therapy. In some embodiments, the monitoring and management module 312 correlates one or more ECG parameters with one or more cardiac parameters for detecting a cardiac performance state. For example, in some embodiments, the ECG parameters are correlated to one or more of pulmonary arterial pressure (PAP) (including the presence of pulmonary hypertension), pulmonary vascular resistance, right ventricular pressure (systolic and/or diastolic), right ventricular hypertrophy (e.g., enlargement of right ventricle, or increase in muscle mass), right ventricular strain, and right ventricular size. In some embodiments, the therapy risk prediction module 310 and/or the monitoring and management module 312 use the ECG data and/or data from one or more of the clinical biomarker module 302, imaging data module 304, and clinical data module 306 to correlate the one or more cardiac parameters relating to a therapy effect and/or cardiac performance state.

In some embodiments, the clinical biomarker module 302 is configured to obtain and optionally store data relating to one or more clinical biomarkers in the subject. In some embodiments, the one or more clinical biomarkers comprise one or more blood borne protein measures, one or more blood borne molecular measures, urine protein, and/or one or more other molecular measures. Exemplary blood borne protein measurements include a B-type natriuretic peptide (BNP), N-terminal(NT)-pro hormone BNP (NT-proBNP), cardiac troponin, or a combination thereof. In some embodiments, said data of one or more clinical biomarkers may include an amount, concentration, and/or level of said one or more clinical biomarkers.

Such clinical biomarkers can correlate to several factors for cardiac health. For example, blood borne measures of cardiac wall stress (NT pro BNP) and myocardial injury (troponin) as well as lipid profiles and diabetic parameters (blood glucose and HbAlc) as well as inflammation (hs CRP) are well established in cardiovascular evaluation and management.

In some embodiments, the data of the one or more clinical biomarkers are obtained via a blood sample from the subject, wherein the blood sample is further processed to identify said one or more clinical biomarkers. In some embodiments, the blood sample can be obtained by a healthcare provider (e.g., a medical and/or health professional as described herein), and/or by the subject or a non-medical or non-health professional. In some embodiments, the blood sample can be processed in a laboratory, hospital, medical clinic, health center, or any combination thereof. In some embodiments, the blood sample can be processed using a point of care device, thereby enabling the subject to have the blood sample processed in a non-medical location (for example at home). In some embodiments, the results of the blood sample processing identifies the one or more clinical biomarkers (and relative data, such as amount, concentration and/or level), wherein said results can be inputted or sent to a computing device in operative communication with the clinical biomarker module 302, thereby enabling the clinical biomarker module 302 to receive the one or more clinical biomarkers. In some embodiments, the results of the blood processing are inputted or sent directly to the clinical biomarker module 302.

In some embodiments, the imaging data module 304 is configured to obtain and optionally store images obtained pertaining to the subject. In some embodiments, such images comprise MRI scans (for example, cardiac MRI scans), and/or echocardiogram scans. In some embodiments, the images are sent from a healthcare provider (e.g., from a medical or other healthcare clinic), via a computing device in operative communication with the imaging data module 304. In some embodiments, the subject or other non-medical professional is configured to upload or send the image(s) to be received by the imaging data module 304.

In some embodiments, the imaging data module 304 is configured to extract one or more echocardiography parameters. For example, exemplary left ventricle echocardiography parameters include outflow track obstruction, ejection fraction, fractional shortening, mass index, maximum wall thickness, septal thickness, and/or strain. Exemplary mitral value echocardiography parameters include systolic anterior motion and/or regurgitation.

Exemplary left atrium echocardiography parameters include maximal and/or minimal diameter, volume index, ejection fraction, function and/or strain. Exemplary right ventricle echocardiography parameters include mass index, wall thickness, strain, right ventricle ejection fraction, and/or right ventricle systolic pressure (e.g., as measured by tricuspid regurgitation velocity Doppler).

In some embodiments, the clinical data module 306 is configured to obtain and optionally store one or more clinical parameters. FIG. 3B provide exemplary clinical parameters. For example, in some embodiments, the clinical parameters comprise the subject's age, sex, weight, body mass index, height, etc., In some embodiments, clinical parameters (e.g., weight) are communicated to the clinical data module 306 via a smart device, such as a weight scale in operative communication with the clinical data module 306. In some cases, weight is an important factor for cardiovascular care, both as chronic and acute risk measure. Chronically weight may serve as an indirect correlate of cardiovascular fitness and is often measured in routine clinical practice. In some cases, acute changes in weight are primarily related to fluid and increases may be signs of worsening heart failure. In some cases, daily home weight measures are employed in heart failure management for select patients.

In some embodiments, the clinical parameters include physiological data, e.g., heart rate, blood glucose, blood pressure, respiration rate, body temperature, blood volume, blood oxygen saturation, etc. In some embodiments, such physiological data is obtained via a smart device, such as a wearable device or other device configured to be in operative communication with the clinical data module 306.

In some embodiments, the clinical parameters comprise subject exercise results, subject activity (for example, fitness activity or other movement), velocity, sleep data, etc. In some embodiments, such clinical parameters are obtained via a smart device, such as a wearable device or other device configured to be in operative communication with the clinical data module 306. For example, in some embodiments, an accelerometer is used to provide such clinical parameters.

In some embodiments, exercise testing is important in assessing cardiopulmonary conditions. In some embodiments, exercise testing measurements include maximal exercise in power (METs/Watt) and distance (for example, a six minute walk). In some embodiments, maximal exercise testing on a treadmill or stationary cycle is conducted in combination with ECG monitoring and often with an imaging to assess cardiac function and/or ischemia. In some embodiments, distance testing is conducted on a defined course under supervision with or without ECG monitoring.

Connected consumer wearable technologies are well established in their ability to measure multiple ambulatory activity parameters including total steps, velocity and duration. These data can be adapted to provide clinical insights similar to supervised exercise protocols, especially when integrated ECG and other ambulatory data.

In some embodiments, any one of such clinical parameters can be inputted by the subject and/or a health care provider.

In some embodiments, the clinical parameters include other data that can be inputted by the subject and/or healthcare provider, or transmitted from a healthcare provider. For example, such other data can include reported symptoms by the subject, past medical history, family medical history, genetics, or any combination thereof.

In some embodiments, the assessment of symptoms is an important factor for cardiovascular care. In some embodiments, symptoms are primarily recorded during patient encounters while patients/families may notify healthcare providers to changes/lack of changes ad hoc. In some cases, symptom logs are not commonly employed outside of the specific testing protocols or the clinical trial setting. As described herein, in some embodiments, the cardiac performance tool 206 is configured to receive inputting of symptoms by the subject, healthcare provider, or other individual.

Connected consumer wearable technologies are well suited to prompt individuals to input the symptoms with both structured and open fields. These inputs can be integrated with other ambulatory inputs, providing a longitudinal record of symptoms with associated clinical parameters.

### Decision Engine(s) and Decision Engine Data

In some embodiments, the decision engine module 308 applies one or more algorithms to predict an efficacy of a therapy in reducing an abnormality in a cardiac performance (e.g., determines an effect of the therapy on the cardiac performance abnormality), and/or to determine a cardiac performance state. In some embodiments, such one or more algorithms utilizes one or more of the subject health parameters (as described herein) to predict such an efficacy and/or cardiac performance state. As described herein, such subject health parameters may be obtained via ECG data, clinical biomarkers, imaging data, and/or clinical data parameters (as described herein). In some embodiments, one or more algorithms may correspond to determining the efficacy of a specific therapy in reducing the abnormality of a cardiac performance. As described herein, in some embodiments, the therapy comprises a drug therapy, such as administering a myosin inhibitor, a PDE5 inhibitor, one or more beta blockers, one or more calcium channel blockers, one or more heart rhythm drugs, and/or one or more blood thinners. IN some embodiments, one or more algorithms may correspond to determining a cardiac performance state, including detecting an abnormality with the cardiac performance.

In some embodiments, the one or more decision engines apply algorithms (e.g., algorithms embodied in trained models) to correlate the various combinations of subject health parameters of the subject with a given therapy and/or a cardiac performance state. In some embodiments, at least one of the one or more algorithms may comprise a machine learning algorithm incorporating artificial intelligence (AI) to help improve accuracy of said prediction of efficacy, and/or for determining a cardiac performance state. For example, with respect to determining a treatment efficacy, in some embodiments, said artificial intelligence is applied to trained model data (which may be included in the decision engine data) and/or data including subject health parameters from a plurality of individuals (e.g., a cohort of individuals) with one or more therapy effects, so as to identify correlations between said subject health parameters and a therapy effect, thereby training the model. In some embodiments, the therapy effect corresponds to a reduced cardiac performance (e.g., a negative effect), an improved cardiac performance (e.g., a positive effect), or no change to the cardiac performance (e.g., no effect).

In some embodiments, any one of the decision engine(s) described herein is any one of a regression model (e.g., linear regression, logistic regression, or polynomial regression), decision tree, random forest, gradient boosted machine learning model, support vector machine, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof. In particular embodiments, any one of the decision engine(s) described herein is a logistic regression model. In particular embodiments, any one of the decision engine(s) described herein is a random forest classifier. In particular embodiments, any one of the decision engine(s) described herein is a gradient boosting model.

In some embodiments, any one of the decision engine(s) described herein (e.g., a trained model) can be trained using a machine learning implemented method, such as any one of a linear regression algorithm, logistic regression algorithm, decision tree algorithm, support vector machine classification, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In particular embodiments, the machine learning implemented method is a logistic regression algorithm. In particular embodiments, the machine learning implemented method is a random forest algorithm. In particular embodiments, the machine learning implemented method is a gradient boosting algorithm, such as XGboost. In some embodiments, any one of the trained model(s) described herein is trained using supervised learning algorithms, unsupervised learning algorithms, semi-supervised learning algorithms (e.g., partial supervision), weak supervision, transfer, multi-task learning, or any combination thereof.

In some embodiments, any one of the trained model(s) described herein has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, node values in a decision tree, and coefficients in a regression model. The model parameters of the risk prediction model are trained (e.g., adjusted) using the training data to improve the predictive capacity of the risk prediction model.

In some embodiments, any one of the trained model(s) described herein are trained via training data located in the trained model data (which may be included with the decision engine module 318). In some embodiments, such trained model data includes subject health parameters obtained from a plurality of individuals, and corresponding therapies administered to said individuals along with the effect on a cardiac performance abnormality. In some embodiments, such trained model data includes subject health parameters from a plurality of individuals, such as one or more cardiac parameters as described herein, and corresponding cardiac performance state.

In various embodiments, the training data used for training any one of the trained model(s) described herein includes reference ground truths that indicate that a therapy was associated with a negative effect, positive effect, or no effect on a cardiac performance abnormality, (hereafter also referred to as “negative” or “−” “positive” or “+”, or “neutral”). In various embodiments, the reference ground truths in the training data are binary values, such as “1” or “0 .” For example, a therapy that was diagnosed with a positive effect on a cardiac performance abnormality can be identified in the training data with a value of “1” whereas a therapy that was diagnosed with a negative effect on a cardiac performance abnormality can be identified in the training data with a value of “0 .” In various embodiments, any one of the trained model(s) described herein are trained using the training data to minimize a loss function such that any one of the trained model(s) described herein can better predict the outcome (e.g., future diagnosis of a cardiac condition) based on the input (e.g., extracted features of the subject's health parameters). In some embodiments, the loss function is constructed for any of a least absolute shrinkage and selection operator (LASSO) regression, Ridge regression, or ElasticNet regression. In some embodiments, any one of the trained model(s) described herein is a random forest model, and is trained to minimize one of Gini impurity or Entropy metrics for feature splitting, thereby enabling any one of the trained model(s) described herein to more accurately predict an effect of a therapy on a cardiac performance abnormality.

In various embodiments, the training data can be obtained and/or derived from a publicly available database. In some embodiments, the training data can be obtained and collected independent of publicly available databases. Such training data can be a custom dataset. As described herein, in some embodiments, the training data includes subject health parameters (as described herein) from a plurality of individuals (e.g., a cohort of individuals), wherein an efficacy of at least one therapy on a cardiac performance abnormality is identified for each individual based on the corresponding subject health parameters of the individual.

In some embodiments, correlations relating the subject health parameters with one or more therapies as obtained via the system 200 is stored in the decision engine data storage 318. In some embodiments, decision engine data storage includes ranges and/or combinations that correlate the subject health data (e.g., cardiac parameters, ECG data, imaging, clinical biomarkers, subject health parameters) with a particular therapy efficacy on a cardiac performance abnormality, and/or a cardiac performance state. For example, in some cases, certain subject health parameters correlate a therapy with a negative effect on a cardiac performance, such that a decision engine incorporating said subject health parameters received from the subject with stored correlations provide a measure of risk relating to a magnitude of likelihood and/or a severity of a negative effect occurring. In some embodiments, the decision engine data storage 312 is updated via communication with an external database, and/or is updated based on subject health parameters as received from the subject.

### Therapy Risk Prediction

In some embodiments, as described herein, the therapy risk prediction module 310 is configured to predict an efficacy of a therapy on a cardiac performance abnormality. In some embodiments, the therapy risk prediction module 310 is configured to apply one or more decision engines with one or more of the subject's health parameters (as described herein) to identify a therapy as i) having a negative effect on a cardiac performance abnormality, and optionally a risk level correlating to a likelihood and/or severity of the cardiac performance abnormality worsening, ii) having no effect on the cardiac performance abnormality, or iii) having a positive effect on the cardiac performance abnormality. As described herein, in some embodiments, the one or more decision engines correlates the subject health parameter data from a plurality of individuals and corresponding effect of a therapy on a cardiac performance abnormality to generate said correlations.

In some embodiments, the therapy risk prediction module 310 is configured to communicate to the subject and/or healthcare provider (as described herein) regarding a determined efficacy of a therapy, so as to help decided whether a therapy is to be administered on the subject.

### Monitoring and Management of a Cardiac Health Status

In some embodiments, the monitoring and management (MM) module 312 is configured to monitor the cardiac performance state of a subject via periodic measurement and input of health parameters. In some embodiments, such monitoring occurs at steps 504 and/or 510 of FIG. 1. For example, in some embodiments, the MM module 312, in communication with a device configured to provide a subject health parameter, such as an ECG device or an accelerometer, is configured to obtain data from such devices and apply one or more decision engines (as described herein) to determine a cardiac performance state.

In some embodiments, the MM module 312 first establishes a baseline evaluation of the cardiac performance state of the subject, based on health parameters initially received by the system 200. In some embodiments, the baseline evaluation establishes a benchmark for the subject's cardiac performance state for future monitoring (e.g., step 510 of FIG. 1), thereby showing a regression or progression of a cardiac performance state (e.g., worsening abnormality).

In some embodiments, as described herein, the MM module 312 is configured to monitor the cardiac health status of the subject via periodic retrieval of some of the health parameters (as described herein) at a prescribed frequency, so as to identify changes to a cardiac performance state (e.g., change in abnormality severity) in a patient, as compared to the baseline evaluation. In some embodiments, the prescribed frequency includes any temporal frequency prescribed by the subject, a medical professional, or other individual. For example, in some embodiments, the prescribed frequency includes obtaining one or more health parameters daily, every 2, 3, 4, 5, or 6 days, weekly, bi-weekly, monthly, every 4 to 20 weeks, etc. In some embodiments, prompts are provided for a subject to go into a health clinic (e.g., hospital, medical clinic, etc., to obtain one or more subject health parameters, such as an echocardiogram, blood draw, etc.).

In some embodiments, periodic monitoring by the MM module 312 enables for reduced the risk of the subject developing systolic dysfunction and/or having a reduced cardiac output, as described herein.

### Intervention Recommendations

In some embodiments, the intervention module 314 is configured to recommend an adjustment to an existing therapy, as described herein (for example, see step 514 of FIG. 1). In some embodiments, the intervention module 314 is configured to obtain a cardiac performance state determined by the MM module 312, and determine a change from a previous cardiac performance state. In some embodiments, the MM module 312 is configured to automatically recommend a change in a therapy administered based on a negative effect on a cardiac performance state (e.g., worsening abnormality in the cardiac performance), or base on observing no positive effect on a cardiac performance state (e.g., no improvement in a cardiac performance abnormality). In some embodiments, the intervention module 314 alerts the subject and/or a healthcare provider (as described herein) regarding a change to the cardiac performance state, and is configured to receive a recommendation for a therapy adjustment. In some embodiments, the intervention module 314 communicates a recommendation to the therapy risk prediction module to determine an efficacy of the recommended therapy, so as to alert the subject and/or healthcare provider of whether such a therapy should be administered.

In some embodiments, adjusting the therapy comprises maintaining the same therapy, but changing one or more of the therapy dosage and the frequency. For example, where a myosin inhibitor is provided, in some embodiments, adjusting the therapy comprises providing a reduced amount of the myosin inhibitor, and/or reducing the frequency of when the myosin inhibitor is provided.

In some embodiments, adjusting the therapy comprises changing one or more of the therapy dosage and the frequency, and/or providing one or more additional therapies. For example, where a myosin inhibitor is provided and found to reduce the contractile force of the right ventricle while the PAP remains elevated, a PDE5 inhibitor or other therapy to decrease the pulmonary vascular resistance is recommended to be administered to the subject.

In some embodiments, adjusting the therapy comprises to stop providing the existing therapy, and recommend one or more additional therapies to be provided, or alternatively, not provide any therapy (either for a prescribed duration, or stop completely).

In some embodiments, providing one or more additional therapies, either with or without the existing therapy (from step 506 from FIG. 1), includes first predicting the effect of the therapy prior to either recommending or administering to the subject (via step 508 from FIG. 1). In some embodiments, where a negative effect to the cardiac performance is detected, or in some cases, a positive effect is not identified, the method re-starts after step 504.

### Communication

In some embodiments, the communication module 316 is configured to communicate with a healthcare provider, to send alerts and/or cardiac health statuses, and/or relay to the subject or the cardiac performance tool 206 (and associated modules) recommendations, data (such as additional health parameters, including clinical biomarkers and/or images), and messages to the subject.

In some embodiments, as described herein, the cardiac performance tool 206 is configured to display the subject's health parameters, cardiac performance state, and efficacy of a therapy using a display interface (e.g., a monitor, screen, etc.).

### IV. Computer Implementation

The methods described herein, including the methods of implementing one or more decision engines for determining a cardiac performance state and/or efficacy of a therapy on a cardiac performance abnormality, are, in some embodiments, performed on one or more computers.

For example, the building and deployment of any method described herein can be implemented in hardware or software, or a combination of both. In one embodiment, a machine-readable storage medium is provided, the medium comprising a data storage material encoded with machine readable data which, when using a machine programmed with instructions for using said data, is capable of executing any one of the methods described herein and/or displaying any of the datasets or results (e.g., cardiac performance state, risk prediction) described herein. Some embodiments can be implemented in computer programs executing on programmable computers, comprising a processor and a data storage system (including volatile and non-volatile memory and/or storage elements), and optionally including a graphics adapter, a pointing device, a network adapter, at least one input device, and/or at least one output device. A display may be coupled to the graphics adapter.

Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of an embodiment. The databases of some embodiments can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g., word processing text file, database format, etc.

In some embodiments, the methods described herein, including the methods for determining a cardiac health status, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 4 illustrates an example computer for implementing the entities shown in FIGS. 1-3. The computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input device 414, and network adapter 416 are coupled to the I/O controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of pointing device, a keyboard, or some combination thereof, and is used to input data into the computer 400. In some embodiments, the computer 400 may be configured to receive input (e.g., commands) from the input interface 414 via gestures from the user. The network adapter 416 couples the computer 400 to one or more computer networks.

The graphics adapter 412 displays images and other information on the display 418. In various embodiments, the display 418 is configured such that the user may (e.g., subject, healthcare professional, non-healthcare professional) may input user selections on the display 418 to, for example, initiate the system for determining a cardiac health status. In one embodiment, the display 418 may include a touch interface. In various embodiments, the display 418 can show a cardiac performance state, a predicted therapy efficacy, and alerts relating to a cardiac performance abnormality.

The computer 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computers 400 used by the entities of FIGS. 1-3 can vary depending upon the embodiment and the processing power required by the entity. For example, the cardiac performance tool 206 can run in a single computer 400 or multiple computers 400 communicating with each other through a network such as in a server farm. The computers 400 can lack some of the components described above, such as graphics adapters 412, and displays 418.

### V. Systems

Further disclosed herein are systems for implementing one or more decision engines for determining a cardiac performance state and/or determining an efficacy of a therapy on a cardiac performance abnormality. In various embodiments, such a system can include at least the cardiac performance tool 206 described above in FIG. 2. In some embodiments, the cardiac performance tool 206 is embodied as a computer system, such as a computer system with example computer 400 described in FIG. 4.

In some embodiments, the system includes one or more auxiliary devices, such as an ECG device, an ambulatory monitoring device, an imaging device, an accelerometer, a weight scale, etc., or any combination thereof, as described herein. In some embodiments, the system includes both the cardiac health tool 206 (e.g., a computer system) and one or more of the auxiliary devices. In such embodiments, the cardiac performance tool 206 can be communicatively coupled with any combination of the auxiliary devices to receive data therefrom.

All publications, patents, patent applications and other documents cited in this application are hereby incorporated by reference herein in their entireties for all purposes to the same extent as if each individual publication, patent, patent application or other document were individually indicated to be incorporated by reference for all purposes.

### VI. Numbered Embodiments

Embodiment 1: A method for treating hypertrophic cardiomyopathy in a subject, the method comprising: detecting an abnormality with a cardiac performance in the subject, wherein the abnormality corresponds to one or more of an abnormal pulmonary arterial pressure, pulmonary hypertension, abnormal right ventricular pressure, right ventricular hypertrophy, right ventricular strain, and right ventricular size; optionally predicting an efficacy of a first therapy for reducing the abnormality; administering the first therapy or a second therapy on the subject, optionally based on the predicted efficacy of the first therapy; and monitoring the abnormality after administering the first therapy or the second therapy on the subject, and optionally detecting a negative effect.
Embodiment 2: The method of Embodiment 1, wherein the first and/or second therapy comprises one or more types of therapy comprising myosin inhibition, a therapy for reducing pulmonary vascular resistance, one or more beta blockers, one or more calcium channel blockers, one or more heart rhythm drugs, and/or one or more blood thinners, or any combination thereof.
Embodiment 3: The method of Embodiment 1 or 2, wherein the negative effect comprises an increased abnormality in the cardiac performance from (a).
Embodiment 4: The method of any one of Embodiments 1 to 3, wherein predicting the efficacy of the first therapy comprises: obtaining one or more health parameters of the subject; and applying the one or more health parameters to one or more correlations relating to the first therapy.
Embodiment 5: The method of Embodiment 4, wherein the one or more correlations is based on: one or more health parameters received from a cohort of individuals; and for each individual of the cohort of individuals, an i) negative effect to a corresponding cardiac performance due to the first therapy being administered, ii) a positive result to the corresponding cardiac performance due to the first therapy being administered, or iii) no effect to the corresponding cardiac performance due to the first therapy being administered, such that at least one of the one or more health parameters (from a corresponding individual of the cohort of individuals) is correlated with an effect on a cardiac performance when combined with administration of the first therapy.
Embodiment 6: The method of Embodiment 4 or 5, wherein applying the one or more correlations of data comprises using a machine learning algorithm.
Embodiment 7: The method of any one of Embodiments 1 to 6, wherein detecting the abnormality and/or monitoring the abnormality comprises using an implanted pulmonary arterial monitor, obtaining an echocardiogram, obtaining an electrocardiogram (ECG), obtaining one or more biomarkers, or a combination thereof.
Embodiment 8: The method of Embodiment 7, wherein detecting the abnormality and/or monitoring the abnormality is conducted in a medical setting, in an ambulatory setting, or both.
Embodiment 9: The method of Embodiment 7 or 8, wherein detecting the abnormality and/or monitoring the abnormality comprises obtaining an ECG, wherein the ECG comprises a single lead ECG, a 2 lead ECG, a 6 lead ECG, or a 12 lead ECG.
Embodiment 10: The method of any one of Embodiments 1 to 9, further comprising adjusting the first or second therapy based on detecting a corresponding negative effect or corresponding no effect on the abnormality in the cardiac performance.
Embodiment 11: The method of Embodiment 10, wherein adjusting the first or second therapy comprises administering a third therapy, and/or reducing an amount of the first or second therapy administered.
Embodiment 12: The method of Embodiment 11, wherein reducing the amount of the first or second therapy administered comprises reducing a frequency of the first or second therapy is administered, and/or reducing a dosage of the first or second therapy administered.
Embodiment 13: The method of any one of Embodiments 1 to 12, wherein prior to detecting the cardiac performance abnormality, further comprising administering an initial HCM treatment on the subject.
Embodiment 14: The method of Embodiment 13, wherein the initial HCM treatment comprises administering a myosin inhibitor to the subject.
Embodiment 15: The method of any one of Embodiments 1 to 14, wherein the hypertrophic cardiomyopathy comprises an obstructive hypertrophic cardiomyopathy (oHCM).
Embodiment 16: The method of Embodiment 15, wherein prior to detecting the cardiac performance abnormality, further comprising at least partially removing an obstruction relating to the oHCM.
Embodiment 17: The method of Embodiment 16, wherein the at least partially removing the obstruction comprises performing a septal myectomy.
Embodiment 18: A non-transitory computer readable medium for treating hypertrophic cardiomyopathy in a subject, the non-transitory computer readable medium comprising instructions that, when executed by a processor, cause the processor to perform operations including: detecting an abnormality with a cardiac performance in the subject, wherein the abnormality corresponds to one or more of an abnormal pulmonary arterial pressure, pulmonary hypertension, abnormal right ventricular pressure, right ventricular hypertrophy, right ventricular strain, and right ventricular size; optionally predicting an efficacy of a first therapy for reducing the abnormality; determining a treatment comprising an administration of the first therapy or a second therapy on the subject, optionally based on the predicted efficacy of the first therapy; and monitoring the abnormality after administering the first therapy or the second therapy on the subject, and optionally detecting a negative effect.
Embodiment 19: The non-transitory computer readable medium of Embodiment 18, wherein the first and/or second therapy comprises one or more types of therapy comprising myosin inhibition, a therapy for reducing pulmonary vascular resistance, one or more beta blockers, one or more calcium channel blockers, one or more heart rhythm drugs, and/or one or more blood thinners, or any combination thereof.
Embodiment 20: The non-transitory computer readable medium of Embodiment 18 or 19, wherein the negative effect comprises an increased abnormality in the cardiac performance from (a).
Embodiment 21: The non-transitory computer readable medium of any one of Embodiments 18 to 20, wherein predicting the efficacy of the first therapy comprises:
   obtaining one or more health parameters of the subject; and applying the one or more health parameters to one or more correlations relating to the first therapy.
Embodiment 22: The non-transitory computer readable medium of Embodiment 21, wherein the one or more correlations is based on: one or more health parameters received from a cohort of individuals; and for each individual of the cohort of individuals, an i) negative effect to a corresponding cardiac performance due to the first therapy being administered, ii) a positive result to the corresponding cardiac performance due to the first therapy being administered, or iii) no effect to the corresponding cardiac performance due to the first therapy being administered, such that at least one of the one or more health parameters (from a corresponding individual of the cohort of individuals) is correlated with an effect on a cardiac performance when combined with administration of the first therapy.
Embodiment 23: The non-transitory computer readable medium of Embodiment 21 or 22, wherein applying the one or more correlations of data comprises using a machine learning algorithm.
Embodiment 24: The non-transitory computer readable medium of any one of Embodiments 18 to 23, wherein detecting the abnormality and/or monitoring the abnormality comprises obtaining one or more cardiac parameters of the subject using an implanted pulmonary arterial monitor, obtaining an echocardiogram, obtaining an electrocardiogram (ECG), obtaining one or more biomarkers, or a combination thereof.
Embodiment 25: The non-transitory computer readable medium of Embodiment 24, wherein detecting the abnormality and/or monitoring the abnormality is conducted in a medical setting, in an ambulatory setting, or both.
Embodiment 26: The non-transitory computer readable medium of Embodiment 24 or 25, wherein detecting the abnormality and/or monitoring the abnormality comprises obtaining an ECG, wherein the ECG comprises a single lead ECG, a 2 lead ECG, a 6 lead ECG, or a 12 lead ECG.
Embodiment 27: The non-transitory computer readable medium of any one of Embodiments 18 to 26, further comprising determining an adjustment of the first or second therapy based on detecting a corresponding negative effect or corresponding no effect on the abnormality in the cardiac performance.
Embodiment 28: The non-transitory computer readable medium of Embodiment 27, wherein the adjustment of the first or second therapy comprises determining an administration of a third therapy, and/or reducing an amount of the first or second therapy administered.
Embodiment 29: The non-transitory computer readable medium of Embodiment 28, wherein reducing the amount of the first or second therapy administered comprises reducing a frequency of the first or second therapy is administered, and/or reducing a dosage of the first or second therapy administered.
Embodiment 30: The non-transitory computer readable medium of any one of Embodiments 18 to 29, wherein prior to detecting the cardiac performance abnormality, an initial HCM treatment is administered on the subject.
Embodiment 31: The non-transitory computer readable medium of Embodiment 30, wherein the initial HCM treatment comprises administering a myosin inhibitor to the subject.
Embodiment 32: The non-transitory computer readable medium of any one of Embodiments 18 to 31, wherein the hypertrophic cardiomyopathy comprises an obstructive hypertrophic cardiomyopathy (oHCM).
Embodiment 33: The non-transitory computer readable medium of Embodiment 32, wherein prior to detecting the cardiac performance abnormality, an obstruction relating to the oHCM is at least partially removed.
Embodiment 34: The non-transitory computer readable medium of Embodiment 33, wherein the obstruction is at least partially removed via a septal myectomy.
Embodiment 35: A system for treating hypertrophic cardiomyopathy in a subject, the subject comprising: one or more processors; and one or more memories storing instructions that, when executed by the one or more processors, cause the system to perform operations including: detecting an abnormality with a cardiac performance in the subject, wherein the abnormality corresponds to one or more of an abnormal pulmonary arterial pressure, pulmonary hypertension, abnormal right ventricular pressure, right ventricular hypertrophy, right ventricular strain, and right ventricular size; optionally predicting an efficacy of a first therapy for reducing the abnormality; determining a treatment comprising an administration of the first therapy or a second therapy on the subject, optionally based on the predicted efficacy of the first therapy; and monitoring the abnormality after administering the first therapy or the second therapy on the subject, and optionally detecting a negative effect.
Embodiment 36: The system of Embodiment 35, wherein the first and/or second therapy comprises one or more types of therapy comprising myosin inhibition, a therapy for reducing pulmonary vascular resistance, one or more beta blockers, one or more calcium channel blockers, one or more heart rhythm drugs, and/or one or more blood thinners, or any combination thereof.
Embodiment 37: The system of Embodiment 35 or 36, wherein the negative effect comprises an increased abnormality in the cardiac performance from (a).
Embodiment 38: The system of any one of Embodiments 35 to 37, wherein predicting the efficacy of the first therapy comprises: obtaining one or more health parameters of the subject; and applying the one or more health parameters to one or more correlations relating to the first therapy.
Embodiment 39: The system of Embodiment 38, wherein the one or more correlations is based on: one or more health parameters received from a cohort of individuals; and for each individual of the cohort of individuals, an i) negative effect to a corresponding cardiac performance due to the first therapy being administered, ii) a positive result to the corresponding cardiac performance due to the first therapy being administered, or iii) no effect to the corresponding cardiac performance due to the first therapy being administered, such that at least one of the one or more health parameters (from a corresponding individual of the cohort of individuals) is correlated with an effect on a cardiac performance when combined with administration of the first therapy.
Embodiment 40: The system of Embodiment 38 or 39, wherein applying the one or more correlations of data comprises using a machine learning algorithm.
Embodiment 41: The system of any one of Embodiments 35 to 40, wherein detecting the abnormality and/or monitoring the abnormality comprises obtaining one or more cardiac parameters of the subject using an implanted pulmonary arterial monitor, obtaining an echocardiogram, obtaining an electrocardiogram (ECG), obtaining one or more biomarkers, or a combination thereof.
Embodiment 42: The system of Embodiment 41, wherein detecting the abnormality and/or monitoring the abnormality is conducted in a medical setting, in an ambulatory setting, or both.
Embodiment 43: The system of Embodiment 41 or 42, wherein detecting the abnormality and/or monitoring the abnormality comprises obtaining an ECG, wherein the ECG comprises a single lead ECG, a 2 lead ECG, a 6 lead ECG, or a 12 lead ECG.
Embodiment 44: The system of any one of Embodiments 35 to 43, further comprising determining an adjustment of the first or second therapy based on detecting a corresponding negative effect or corresponding no effect on the abnormality in the cardiac performance.
Embodiment 45: The system of Embodiment 44, wherein the adjustment of the first or second therapy comprises determining an administration of a third therapy, and/or reducing an amount of the first or second therapy administered.
Embodiment 46: The system of Embodiment 45, wherein reducing the amount of the first or second therapy administered comprises reducing a frequency of the first or second therapy is administered, and/or reducing a dosage of the first or second therapy administered.
Embodiment 47: The system of any one of Embodiments 35 to 46, wherein prior to detecting the cardiac performance abnormality, an initial HCM treatment is administered on the subject.
Embodiment 48: The system of Embodiment 47, wherein the initial HCM treatment comprises administering a myosin inhibitor to the subject.
Embodiment 49: The system of any one of Embodiments 35 to 48, wherein the hypertrophic cardiomyopathy comprises an obstructive hypertrophic cardiomyopathy (oHCM).
Embodiment 50: The system of Embodiment 49, wherein prior to detecting the cardiac performance abnormality, an obstruction relating to the oHCM is at least partially removed.
Embodiment 51: The system of Embodiment 50, wherein the obstruction is at least partially removed via a septal myectomy.

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

1. A method for treating hypertrophic cardiomyopathy in a subject, the method comprising:
detecting an abnormality with a cardiac performance in the subject, wherein the abnormality corresponds to one or more of an abnormal pulmonary arterial pressure, pulmonary hypertension, abnormal right ventricular pressure, right ventricular hypertrophy, right ventricular strain, and right ventricular size;
optionally predicting an efficacy of a first therapy for reducing the abnormality;
administering the first therapy or a second therapy on the subject, optionally based on the predicted efficacy of the first therapy; and
monitoring the abnormality after administering the first therapy or the second therapy on the subject, and optionally detecting a negative effect.

2. The method of claim 1, wherein the first and/or second therapy comprises one or more types of therapy comprising myosin inhibition, a therapy for reducing pulmonary vascular resistance, one or more beta blockers, one or more calcium channel blockers, one or more heart rhythm drugs, and/or one or more blood thinners, or any combination thereof.

3. The method of claim 1, wherein the negative effect comprises an increased abnormality in the cardiac performance.

4. The method of claim 1, wherein predicting the efficacy of the first therapy comprises: obtaining one or more health parameters of the subject; and applying the one or more health parameters to one or more correlations relating to the first therapy.

5. The method of claim 4, wherein the one or more correlations is based on: one or more health parameters received from a cohort of individuals, and for each individual of the cohort of individuals, a i) negative effect to a corresponding cardiac performance due to the first therapy being administered, ii) a positive result to the corresponding cardiac performance due to the first therapy being administered, or iii) no effect to the corresponding cardiac performance due to the first therapy being administered, such that at least one of the one or more health parameters from an individual of the cohort of individuals is correlated with an effect on a cardiac performance when combined with administration of the first therapy.

6. The method of claim 4, wherein applying the one or more correlations of data comprises using a machine learning algorithm.

7. The method of claim 1, wherein detecting the abnormality and/or monitoring the abnormality comprises using an implanted pulmonary arterial monitor, obtaining an echocardiogram, obtaining an electrocardiogram (ECG), obtaining one or more biomarkers, or a combination thereof.

8. The method of claim 7, wherein detecting the abnormality and/or monitoring the abnormality is conducted in a medical setting, in an ambulatory setting, or both.

9. The method of claim 7, wherein detecting the abnormality and/or monitoring the abnormality comprises obtaining an ECG, wherein the ECG comprises a single lead ECG, a 2 lead ECG, a 6 lead ECG, or a 12 lead ECG.

10. The method of claim 1, further comprising adjusting the first or second therapy based on detecting a corresponding negative effect or corresponding no effect on the abnormality in the cardiac performance.

11. The method of claim 10, wherein adjusting the first or second therapy comprises administering a third therapy, and/or reducing an amount of the first or second therapy administered.

12. The method of claim 11, wherein reducing the amount of the first or second therapy administered comprises reducing a frequency of the first or second therapy is administered, and/or reducing a dosage of the first or second therapy administered.

13. The method of claim 1, wherein prior to detecting the cardiac performance abnormality, further comprising administering an initial HCM treatment on the subject.

14. The method of claim 13, wherein the initial HCM treatment comprises administering a myosin inhibitor to the subject.

15. The method of claim 1, wherein the hypertrophic cardiomyopathy comprises an obstructive hypertrophic cardiomyopathy (oHCM).

16. The method of claim 15, wherein prior to detecting the cardiac performance abnormality, further comprising at least partially removing an obstruction relating to the oHCM.

17. The method of claim 16, wherein the at least partially removing the obstruction comprises performing a septal myectomy.

18. A non-transitory computer readable medium for treating hypertrophic cardiomyopathy in a subject, the non-transitory computer readable medium comprising instructions that, when executed by a processor, cause the processor to perform operations including:
detecting an abnormality with a cardiac performance in the subject, wherein the abnormality corresponds to one or more of an abnormal pulmonary arterial pressure, pulmonary hypertension, abnormal right ventricular pressure, right ventricular hypertrophy, right ventricular strain, and right ventricular size;
optionally predicting an efficacy of a first therapy for reducing the abnormality;
determining a treatment comprising an administration of the first therapy or a second therapy on the subject, optionally based on the predicted efficacy of the first therapy; and
monitoring the abnormality after administering the first therapy or the second therapy on the subject, and optionally detecting a negative effect.

19-34. (canceled)

35. A system for treating hypertrophic cardiomyopathy in a subject, the system comprising: one or more processors; and one or more memories storing instructions that, when executed by the one or more processors, cause the system to perform operations including:
detecting an abnormality with a cardiac performance in the subject, wherein the abnormality corresponds to one or more of an abnormal pulmonary arterial pressure, pulmonary hypertension, abnormal right ventricular pressure, right ventricular hypertrophy, right ventricular strain, and right ventricular size;
optionally predicting an efficacy of a first therapy for reducing the abnormality;
determining a treatment comprising an administration of the first therapy or a second therapy on the subject, optionally based on the predicted efficacy of the first therapy; and
monitoring the abnormality after administering the first therapy or the second therapy on the subject, and optionally detecting a negative effect.

36. The system of claim 35, wherein the first and/or second therapy comprises one or more types of therapy comprising myosin inhibition, a therapy for reducing pulmonary vascular resistance, one or more beta blockers, one or more calcium channel blockers, one or more heart rhythm drugs, and/or one or more blood thinners, or any combination thereof.

37. The system of claim 35, wherein the negative effect comprises an increased abnormality in the cardiac performance.

38. The system of claim 35, wherein predicting the efficacy of the first therapy comprises: obtaining one or more health parameters of the subject; and applying the one or more health parameters to one or more correlations relating to the first therapy.

39. The system of claim 38, wherein the one or more correlations is based on: one or more health parameters received from a cohort of individuals; and for each individual of the cohort of individuals, an i) negative effect to a corresponding cardiac performance due to the first therapy being administered, ii) a positive result to the corresponding cardiac performance due to the first therapy being administered, or iii) no effect to the corresponding cardiac performance due to the first therapy being administered, such that at least one of the one or more health parameters from an individual of the cohort of individuals is correlated with an effect on a cardiac performance when combined with administration of the first therapy.

40. The system of claim 38, wherein applying the one or more correlations of data comprises using a machine learning algorithm.

41. The system of claim 35, wherein detecting the abnormality and/or monitoring the abnormality comprises obtaining one or more cardiac parameters of the subject using an implanted pulmonary arterial monitor, obtaining an echocardiogram, obtaining an electrocardiogram (ECG), obtaining one or more biomarkers, or a combination thereof.

42. The system of claim 41, wherein detecting the abnormality and/or monitoring the abnormality is conducted in a medical setting, in an ambulatory setting, or both.

43. The system of claim 41, wherein detecting the abnormality and/or monitoring the abnormality comprises obtaining an ECG, wherein the ECG comprises a single lead ECG, a 2 lead ECG, a 6 lead ECG, or a 12 lead ECG.

44. The system of claim 35, further comprising determining an adjustment of the first or second therapy based on detecting a corresponding negative effect or corresponding no effect on the abnormality in the cardiac performance.

45. The system of claim 44, wherein the adjustment of the first or second therapy comprises determining an administration of a third therapy, and/or reducing an amount of the first or second therapy administered.

46. The system of claim 45, wherein reducing the amount of the first or second therapy administered comprises reducing a frequency of the first or second therapy is administered, and/or reducing a dosage of the first or second therapy administered.

47. The system of claim 35, wherein prior to detecting the cardiac performance abnormality, an initial HCM treatment is administered on the subject.

48. The system of claim 47, wherein the initial HCM treatment comprises administering a myosin inhibitor to the subject.

49. The system of claim 35 wherein the hypertrophic cardiomyopathy comprises an obstructive hypertrophic cardiomyopathy (oHCM).

50. The system of claim 49, wherein prior to detecting the cardiac performance abnormality, an obstruction relating to the oHCM is at least partially removed.

51. The system of claim 50, wherein the obstruction is at least partially removed via a septal myectomy.
